(19) United States
(12) Patent Application Publication
(10) Pub. No.: US 2024/0325749 A1
(43) Pub. Date: Oct. 3, 2024

(54) TRANSCRANIAL ALTERNATING CURRENT STIMULATION (tACS)

(71) Applicant: Fisher Wallace Laboratories Inc., Brooklyn, NY (US)

(72) Inventor: Kelly Roman, Brooklyn, NY (US)

(21) Appl. No.: 18/624,083

(22) Filed: Apr. 1, 2024

Related U.S. Application Data
(60) Provisional application No. 63/493,312, filed on Mar. 30, 2023.

(51) Int. Cl. A61N 1/36; A61N 1/04

(52) U.S. Cl. CPC A61N 1/36025; A61N 1/0484; A61N 1/36034

(57) ABSTRACT

A method of transcranial alternating current stimulation (tACS) is provided. The method includes generating and transmitting an electrical current to two electrodes contacting a patient's scalp to deliver a therapeutic dose of tACS for a predetermined treatment time. The current has a constant average amplitude of 2.2 mA and a bidirectional square waveform with a carrier of frequency 15 kHz, one modulating waveform of frequency 15 Hz, and another modulating waveform of frequency of 500 Hz. The current includes sub-bursts within a burst duration including a burst-on duration of 50 ms that includes the sub-bursts followed by a burst-off duration of 16.7 ms without the sub-bursts. The current switches polarity after each burst duration. Each sub-burst includes pulses within a sub-burst duration including a sub-burst on duration of 1 ms including the pulses followed by a sub-burst off duration of 1 ms without the pulses. Each pulse extends for about 33.33 μs followed by a pause of 33.33 μs.

### BACKGROUND

Major depressive disorder (MDD) and Generalized Anxiety Disorders (GAD) are highly prevalent; in 2020, 48 million Americans were prescribed antidepressant medications and 49 million were prescribed anxiety treatment medications. Moreover, according to the Centers for Disease Control and Prevention, the rate of clinical depression and anxiety among U.S. adults has more than tripled since the COVID-19 pandemic began; Boston University has reported that as many as 1 in 3 Americans may now suffer from one or both disorders. Approximate 60% of patients with GAD also have MDD, and nearly half of patients with MDD have GAD; this high level of comorbidity makes treatments that can address both conditions simultaneously advantageous. Of the two conditions, MDD is considered more life threatening due to the risk of suicide and the high rate of hospitalizations and other chronic health conditions and disorders, such as substance use disorder, associated with MDD.

For decades, inadequate access to psychiatrists has been recognized as a public health issue for many segments of the U.S. population; the majority of patients with clinical depression who are treated with psychotropic medications receive their prescriptions from their primary care physician rather than a board-certified psychiatrist. However, analyses of primary care practices show a low use of depression care management processes, suggesting that primary care physicians may not be well equipped to effectively manage depression. Some researchers claim that depression is “significantly undertreated,” particularly in the primary care setting. Other research claims that antidepressant medication may be overprescribed to patients who are better suited for lower-risk psychotherapy. There is also far more demand for psychotherapy than there are therapists who can care for patients in a timely manner.

Common antidepressant medications and psychotherapy often take many weeks to be effective. The American Psychiatric Association (APA) Practice Guideline for the Treatment of Patients with Major Depressive Disorder notes that psychotherapy approaches should yield at least moderate improvement (e.g., greater than 20% diminution in symptoms) within 4 to 8 weeks of treatment and recommends that physicians emphasize to patients that 2-4 weeks of medication is needed before beneficial effects may be noticed. This long time period to achieve improvement in symptoms can be problematic, however. Notably, delayed responses to treatment can result in diminished adherence, which affects success of treatment, risk of relapse, and cost of the illness. For example, it has been observed that the first 6 weeks of treatment is a particularly critical period to promote adherence, with increased risks of treatment dropout, relapse, medication discontinuation, vulnerability to suicide, and greater economic burden among those who show early nonadherence to antidepressants. The fear of and experience of side effects also contribute to patients failing to adhere to medications. Less commonly prescribed controlled substances, such as ketamine, and electroconvulsive therapy (ECT) are fast acting, but are extremely expensive to administer, and often cause significant side effects.

Accordingly, there is a need for rapid-acting depression treatments that pose low risk to patients, promote adherence, are self-administered and inexpensive, and may also significantly reduce anxiety.

### SUMMARY

Transcranial Alternating Current Stimulation (tACS) has been investigated as another approach to treat depression. tACS is a form of non-invasive neurostimulation that delivers low dose alternating current to the brain to induce neuroplasticity. Accordingly, tACS has shown promise as an effective for treatment for depression, as well as anxiety, insomnia, and other neuropsychiatric and cognitive disorders.

tACS devices can be self-administered by a patient, and used safely as a standalone treatment or in combination with medications. While regulated as medical devices, tACS devices are not complex or expensive to manufacture. Importantly, there is growing scientific evidence that tACS is safer than medications, and in certain forms, rapid acting.

A challenge facing the development of tACS devices has been discovering a method of delivering electrical current that is simultaneously therapeutic, comfortable for the patient, and able to successfully maintain blinding in a sham controlled clinical trial. If patients find the stimulation to be uncomfortable, they may avoid using the device; if participants in a clinical trial can easily differentiate an active device from a sham device (a device that appears to function normally but does not produce any stimulation), the results of the trial may not be easily defended.

Embodiments of the present disclosure provide systems and methods for improved tACS that are suitable for treatment of depression (e.g., Major Depressive Disorder; MDD), as well as anxiety (e.g., Generalized Anxiety Disorder; GAD), insomnia, and other neuropsychiatric and cognitive disorders. As discussed in greater detail below, a tACS device is provided that has been demonstrated to provide therapeutically effective treatment using a rapidly pulsed, alternating current (AC) output in the form of a bipolar (bi-directional) square waveform that employs a high carrier frequency and modulates to two lower frequencies, with a current amplitude of 2.2 mA (+/−5% manufacturing tolerance). By conducting large-scale, randomized, controlled clinical trials, it has been discovered that this combination of amplitude, pulse rate, waveform and frequency, delivered to the human brain on a daily basis for a predetermined treatment time (e.g., about 20 minutes), twice per day (once soon after the patient wakes, and again before bed), through two electrodes placed in specific locations on either side of the head, provides effective treatment for depression and anxiety. The ability to provide effective therapy at lower current amplitude reduces or substantially eliminates discomfort experienced by patients.

In an embodiment, a method of transcranial alternating current (tACS) stimulation is provided. The method includes generating and transmitting, by a controller including a processor, an electrical current for receipt by two electrodes in contact with opposing lateral sides of a patient's scalp to deliver a therapeutic dose of tACS for a predetermined treatment time. The generated electrical current has a constant, an average amplitude of about 2.2 mA and a bidirectional square waveform utilizing a carrier waveform having a frequency of about 15 kHz, a first modulating waveform having a frequency of about 15 Hz, and a second modulating waveform having a frequency of about 500 Hz. The generated current includes a plurality of sub-bursts within a burst duration, the burst duration including a burst on duration of about 50 ms including the plurality of sub-bursts followed by a burst off duration of about 16.7 ms without the plurality of sub-bursts. The current switches polarity after each burst duration. Each sub-burst includes a plurality of pulses within a sub-burst duration, the sub-burst duration including a sub-burst on duration of 1 ms including the plurality of pulses followed by a sub-burst off duration of about 1 ms without the plurality of pulses. Each pulse of the plurality of pulses extends for about 33.33 μs followed by a pause of about 33.33 μs.

In another embodiment, each of the two electrodes is positioned on the squamous temporal bone, above the posterior aspect of the zygomatic arch and maintained in place during delivery of the therapeutic dose of tACS for the treatment of Major Depressive Disorder (MDD) or Generalized Anxiety Disorder (GAD). The diameter of each of the electrodes is about 36 mm. The method further includes retrieving, by the processor, the predetermined treatment time from a memory in communication with the processor, wherethe predetermined treatment time is about 20 minutes. The method also includes measuring a treatment time during which the therapeutic dose of tACS is delivered to the patient, comparing the treatment time to the predetermined treatment time; and ceasing transmission of the generated electrical current when the elapsed time is equal to the predetermined treatment time.

In a further embodiment, the method further includes administering the therapeutic dose of tACS to the patient twice daily for the predetermined treatment time, one administration occurring within two hours after waking from sleep and another administration occurs within two hours prior to sleeping.

In an embodiment a method for transcranial alternating current (tACS) stimulation is provided. The method includes positioning two electrodes in contact with opposing lateral sides of a patient's scalp. The method further includes generating, by a controller including a processor, an electrical current. The method additionally includes transmitting the generated electrical current to the electrodes to deliver a therapeutic dose of tACS for a predetermined treatment time (e.g., about 20 minutes). The generated electrical current has an amplitude of about 2.2 mA. The generated electrical current further has a waveform including a carrier waveform having a frequency of about 15 kHz, a first modulating waveform having a frequency of about 15 Hz, and a second modulating waveform having a frequency of about 500 Hz.

In another embodiment, each of the two electrodes is positioned on the squamous temporal bone, above the posterior aspect of the zygomatic arch and maintained in place during stimulation.

In another embodiment, each of the two electrodes has a diameter of about 36 mm.

In another embodiment, the therapeutic dose of tACS is sufficient for treatment of at least one of Major Depressive Disorder (MDD) or Generalized Anxiety Disorder (GAD).

In another embodiment, the method further includes retrieving, by the processor, the predetermined treatment time from a memory in communication with the processor. The method also includes measuring a treatment time during which the therapeutic dose of tACS is delivered to the patient. The method additionally includes comparing the treatment time to the predetermined treatment time. The method also includes ceasing transmission of the therapeutic dose of tACS to the patient when the elapsed time is equal to or greater than the predetermined treatment time.

In another embodiment, the waveform is a pulsed, alternating current in the form of a bidirectional square wave.

In another embodiment, the waveform includes a plurality of first square wave sub-bursts having a first polarity that repeats for a burst duration followed by a plurality of second square wave sub-bursts having a second, opposite polarity that repeats for the burst duration.

In another embodiment, the burst duration is about 66.7 ms and includes an on duration and an off duration, wherein the on duration is about 50 ms and extends from the start of the first sub-burst of the burst duration to the end of the last sub-burst of the burst duration, and wherein the off duration is about 16.7 s and extends from the end of the last sub-burst of the burst duration to the end of the burst duration.

In another embodiment, each of the plurality of first sub-bursts and second sub-bursts extends a sub-burst duration of about 2 ms that includes a sub-burst on duration of about 1 ms followed by a sub-burst off duration of about 1 ms.

In another embodiment, each of the first and second plurality of square wave sub-bursts includes a plurality of pulses that extend a pulse duration of about 66.7 ms that includes a pulse on duration of about 33.3 ms followed by a pulse off duration of about 33.3 ms.

In an embodiment, a transcranial alternating current stimulation (tACS) device is provided. The device includes a stimulator including a strap and two electrodes coupled to the strap. The strap is configured to be secured to a human head and position the two electrodes in contact with opposing lateral sides of a patient's scalp. The method also includes a controller including a processor. The controller is in electrical communication with the plurality of electrodes and configured to perform a variety of operations. The controller is configured to generate an electrical current. The controller is further configured to transmit the electrical current to the electrodes to deliver a therapeutic dose of tACS for a predetermined treatment time. The generated electrical current has an amplitude of about 2.2 mA. The generated electrical current has a waveform including a carrier waveform having a frequency of about 15 kHz, a first modulating waveform having a frequency of about 15 Hz, and a second modulating waveform having a frequency of about 500 Hz.

In an embodiment, the strap is configured to maintain each of the two electrodes on the squamous temporal bone, above the posterior aspect of the zygomatic arch.

In an embodiment, each of the two electrodes has a diameter of about 36 mm.

In an embodiment, the controller is further configured to retrieve the predetermined treatment time from a memory in communication with the processor, to measure a treatment time during which the therapeutic dose of tACS is delivered to the patient, to compare the treatment time to the predetermined treatment time; and to cease transmission of the therapeutic dose of tACS to the patient when the elapsed time is equal to or greater than the predetermined treatment time.

In an embodiment, the waveform is a pulsed, alternating current in the form of a bidirectional square wave.

In an embodiment, the waveform includes a plurality of first square wave sub-bursts having a first polarity that repeats for a burst duration followed by a plurality of second square wave sub-bursts having a second, opposite polarity that repeats for the burst duration.

In an embodiment, the burst duration is about 66.7 ms and includes an on duration and an off duration, wherein the on duration is about 50 ms and extends from the start of the first sub-burst of the burst duration to the end of the last sub-burst of the burst duration, and wherein the off duration is about 16.7 s and extends from the end of the last sub-burst of the burst duration to the end of the burst duration.

In an embodiment, each of the plurality of first sub-bursts and second sub-bursts extends a sub-burst duration of about 2 ms that includes a sub-burst on duration of about 1 ms followed by a sub-burst off duration of about 1 ms.

In an embodiment, each of the first and second plurality of square wave sub-bursts includes a plurality of pulses that extend a pulse duration of about 66.7 ms that includes a pulse on duration of about 33.3 ms followed by a pulse off duration of about 33.3 ms.

### DESCRIPTION OF DRAWINGS

These and other features will be more readily understood from the following detailed description taken in conjunction with the accompanying drawings, in which:

FIG. 1 is a diagram illustrating one exemplary embodiment of a treatment environment in which a transcranial alternating current stimulation (tACS) device including a controller and a stimulator is employed to deliver electrical stimulation to a patient's head;

FIG. 2A is a rear-facing perspective view of the stimulator of FIG. 1;

FIG. 2B is a top-down view of the stimulator of FIG. 1;

FIG. 3A is a schematic diagram of an exterior of the controller of FIG. 1;

FIG. 3B is a block diagram illustrating components of the controller of FIG. 1;

FIGS. 4A-4B are schematic illustrations of a constant current waveform generated by the controller for tACS treatment; and

FIG. 5 is a flow diagram illustrating a method of treatment using the tACS device of FIG. 1.

It is noted that the drawings are not necessarily to scale. The drawings are intended to depict only typical aspects of the subject matter disclosed herein, and therefore should not be considered as limiting the scope of the disclosure.

### DETAILED DESCRIPTION

As used herein, a “patient” is a vertebrate, preferably a mammal, more preferably a human. Mammals include, but are not limited to, murines, simians, humans, farm animals, sport animals, and pets.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

Embodiments of systems and corresponding methods for transcranial alternating current stimulation (tACS) are discussed herein. These systems and methods can be employed for treatment of depression (Major Depressive Disorder), as well as anxiety, and sleep maintenance insomnia. However, it can be understood that embodiments of the disclosure can be employed to treat other conditions without limit.

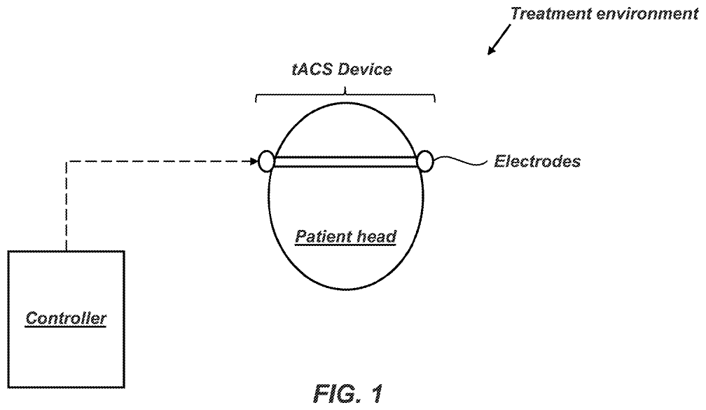

FIG. 1 illustrates a treatment environment in which a tACS device is employed to treat a patient. As shown, the tACS device includes a controller in signal communication with a stimulator. As discussed in greater detail below, the stimulator is configured to couple to a patient's head in order to deliver current to the patient's brain. The controller is configured to allow a user (e.g., patient) to control the tACS device and deliver the electrical current to the electrodes with a predetermined waveform and controlled, constant amplitude for a defined period of time (a predetermined treatment time).

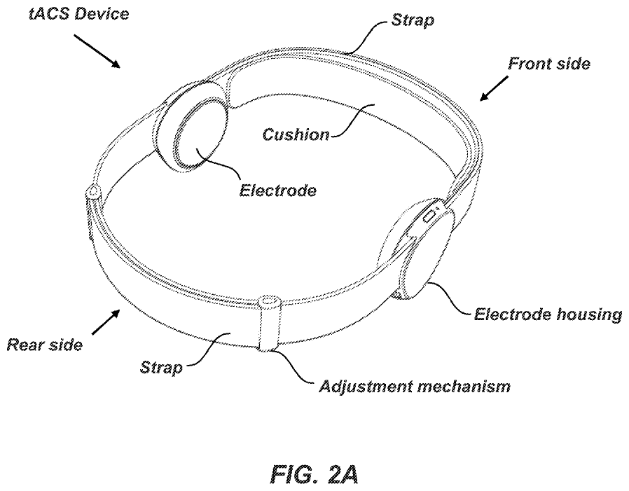

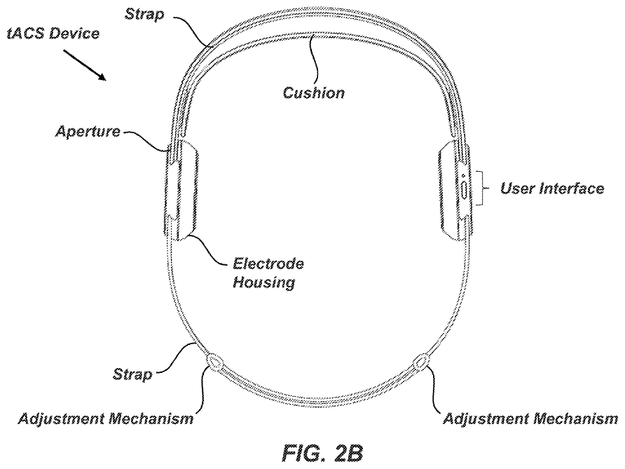

FIG. 2A-2B present the stimulator in a rear-facing view (FIG. 2A) and a top-down view (FIG. 2B). As shown, the stimulator includes a strap and a pair of electrodes, each mounted in an electrode housing that is adjustably secured to the strap.

The strap can be a flexible band that forms a closed loop. In certain embodiments, the strap can be formed from an elastic material capable of stretching to fit around a patient's head, In other embodiments, the strap can include an adjustment mechanism to adjust the circumference of the closed loop in order to snugly fit on the patient's head. The adjustment mechanism can adopt any form allowing the electrodes to be positioned securely in contact with the patient at a selected location (e.g., a strap adjuster, buckles, hook and loop fasteners, snaps, and the like).

In an embodiment, each of the pair of electrodes can be removable from its housing. For example, the electrodes can be sponges. The sponges can have a selected diameter suitable for delivering a therapeutic dose of tACS. For example, the diameter of the electrodes can be about 36 mm (e.g., 36 mm+/−5%). When moistened with water, the sponges conduct electricity via the water. However, it can be appreciated that the electrodes can adopt other configurations that provide equivalent clinical performance to the above-discussed sponges without limit. For example, the electrode can be non-removable from the cavity and formed from a durable, electrically conductive material (e.g., a conductive polymer, metal, etc.)

Each of the pair of electrode housings can be mounted beneath the strap on opposing sides. As shown, the electrode housings can include an aperture extending therethrough (e.g., in a front-back direction). The aperture can be dimensioned to receive the strap therethrough.

In certain embodiments, the position of the receptacle with respect to the strap can be adjusted. Such adjustment is beneficial to ensure that the electrodes are positioned at a correct location of the patient's head.

In one embodiment, this adjustment can be provided by a friction fit between the aperture and the strap. For example, the thickness of the aperture can be slightly smaller than a thickness of the strap to provide the friction fit. When a sliding force sufficient to overcome friction between the electrode housing and the strap is applied, the electrode housing can slide with respect to the strap. When application of the sliding force is stopped, the electrode housing is held in place with respect to the strap.

It can be appreciated that other mechanisms can be employed without limit to secure the electrode housings to the strap and to provide adjustment of the electrode housings with respect to the strap. Examples can include, but are not limited to, reusable adhesives, hook and loop fasteners, snaps, and the like.

Optionally, the stimulator can include a cushion secured to the strap at the front side of the stimulator. So configured, the cushion can contact the patient's forehead when the stimulator is placed on the patient's head, enhancing the patient's comfort during use of the stimulator.

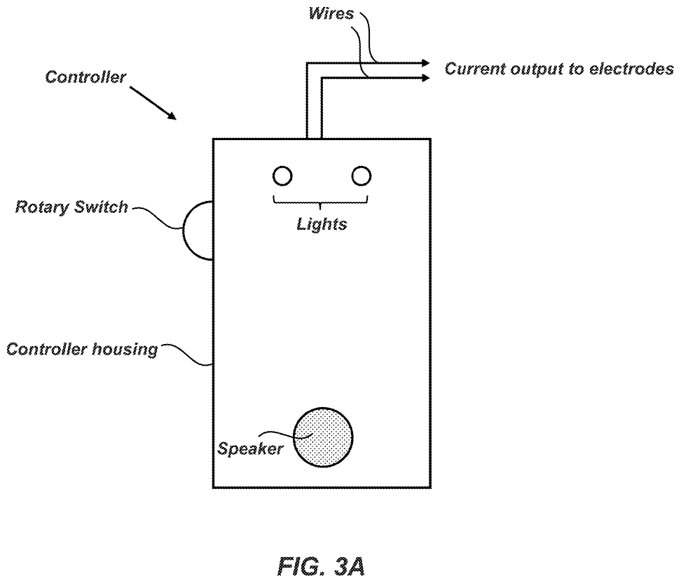

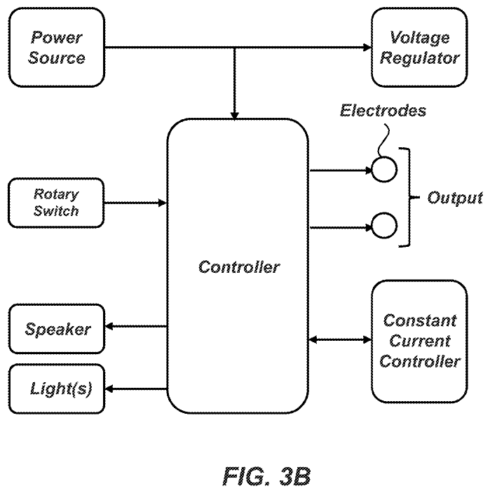

FIGS. 3A-3B illustrate an embodiment of the controller. FIG. 3A illustrates an exterior view of the housing of the controller, while FIG. 3B is a schematic diagram of the electronics of the controller. As shown, the controller includes a power source, a speaker, a constant current controller, a rotary switch, lights, a controller, and a voltage regulator. Each of these components is discussed in greater detail below.

The power source provides power for the entire tACS device. In an embodiment, the power source can be one or more batteries. However, in alternative embodiments, the controller can be configured to receive mains power, after power conditioning.

The rotary switch is configured as power ON/OFF and to start/stop delivery of current to the electrodes for treatment. For example, rotation by one amount from an OFF position to a first position turns on the tACS device and places the device in a standby state. Further rotation to a second position causes delivery of current to the electrodes. In an embodiment, the rotary switch can be a thumb wheel. However, other interface devices (not shown) can be employed without limit. Examples can include, but are not limited to, buttons, sliders, switches, etc.

The controller can further include one or more lights (e.g., LEDs) to indicate status of the tACS device (e.g., OFF, ON, Standby). For example, when the tACS device is on and in a standby mode, one of the lights can be lit and green.

The tACS device is configured to output a constant current. This output is achieved by the constant current controller. According to Ohm's Law, voltage and current are directly related to each other by the resistance of the load. In the context of the tACS device, the load is the human head. The constant current controller addresses the variability in the resistance of the human head. The constant current controller adjusts its own resistance so that the human head plus the controller will produce a fixed current for the output voltage. In this manner, a constant current amplitude of 2.2 mA within a manufacturing tolerance (e.g., +/−5%) can be output using a bidirectional square waveform.

The voltage regulator is an integrated circuit that provides the constant, fixed output voltage in response to commands from the controller, regardless of the change in load or input voltage. This keeps the voltages of the power supply within a range that is compatible with other electrical components of the tACS device.

The speaker is configured to provide audio output to notify the patient/operator that a therapeutic session has concluded or that there is an interrupting issue with the power source. In an embodiment, a buzzer can be used in lieu of a speaker.

The controller includes a processor and can include a memory or be in electrical communication with a memory device (not shown) and the electrode. The controller can power the lights to provide visual confirmation that the tACS device is on. The voltage regulator takes power from the power source and increases the voltage for the output circuitry. The controller produces three frequencies, a carrier frequency and two modulating frequencies and modulates the output using the analog switch. The constant current controller maintains current through the output by use of a resistive feedback circuit which varies its own resistance to that the 2.2 mA+/−5% constant current output is maintained even if there are differences at the output. The controller will drive the speaker/buzzer to indicate the end of a treatment session.

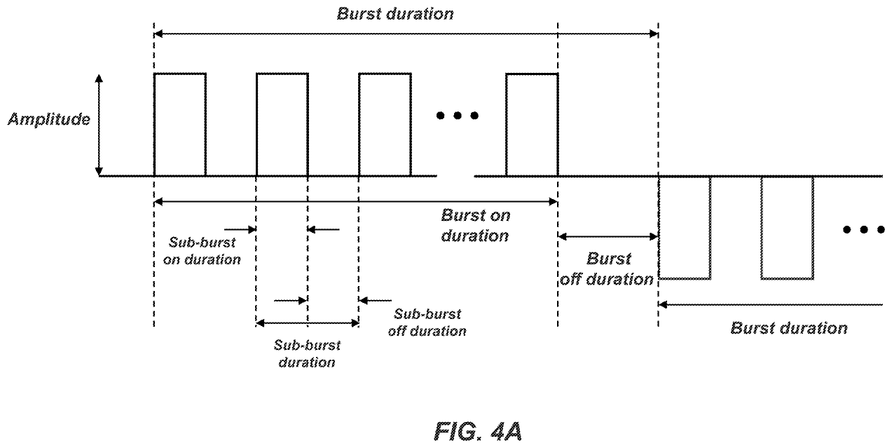

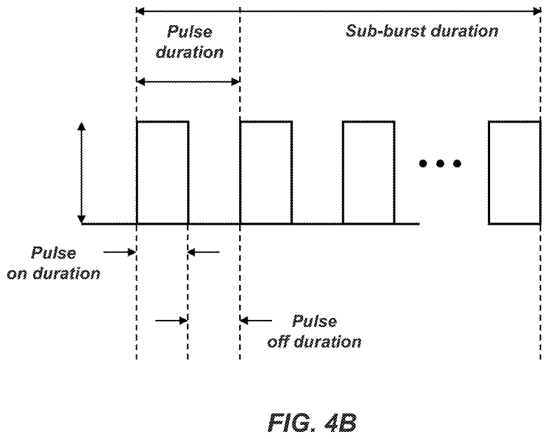

FIGS. 4A-4B are schematic illustrations of the constant current waveform generated by the controller. As shown, the waveform is a pulsed, alternating current in the form of a bidirectional square wave. The parameters of the waveform discussed below can vary within manufacturing tolerances from the stated values (e.g., +/−5%).

As shown in FIG. 4A, The waveform includes a plurality of first square wave sub-bursts having a first polarity that repeats for a burst duration followed by a plurality of second square wave sub-bursts having a second, opposite polarity that repeats for the burst duration.

The burst duration is about 66.7 ms and includes an on duration and an off duration. The on duration is about 50 ms and extends from the start of the first sub-burst of the burst duration to the end of the last sub-burst of the burst duration. The off duration is about 16.7 s and extends from the end of the last sub-burst of the burst duration to the end of the burst duration.

Each of the plurality of first sub-bursts and second sub-bursts extends a sub-burst duration of about 2 ms. The sub-burst duration includes a sub-burst on duration of about 1 ms followed by a sub-burst off duration of about 1 ms.

As further shown in FIG. 4B, each of the first and second plurality of square wave sub-bursts includes a plurality of pulses that extend a pulse duration. The pulse duration is about 66.7 ms and includes a pulse on duration of about 33.3 ms, followed by a pulse off duration of about 33.3 ms.

In the discussion above, the constant current waveform is generated by the controller and transmitted to the electrodes. However, in alternative embodiments, the one or more circuit components of the controller can be positioned in the electrode housing. In certain embodiments, all circuit components of the controller can be housed in the electrode housing. The lights, speaker, and switches of the controller can also be duplicated on the electrode housing. Thus, the tACS device can include a stimulator that combines the functionality of the stimulator and controller of FIG. 1.

So configured, the user can control the tACS device in a variety of ways. In one embodiment, user interface devices (e.g., switches, buttons, etc.) can be provided on the electrode to control the tACS device. In another embodiment, the tACS device can be configured to wirelessly communicate with a computing device (e.g., smartphone, tablet, laptop computer, desktop computer, etc.). The computing device may execute one or more programs that virtually replicate the functionality of physical components of the controller (e.g., the lights, the speaker, and/or switches such as the rotary switch).

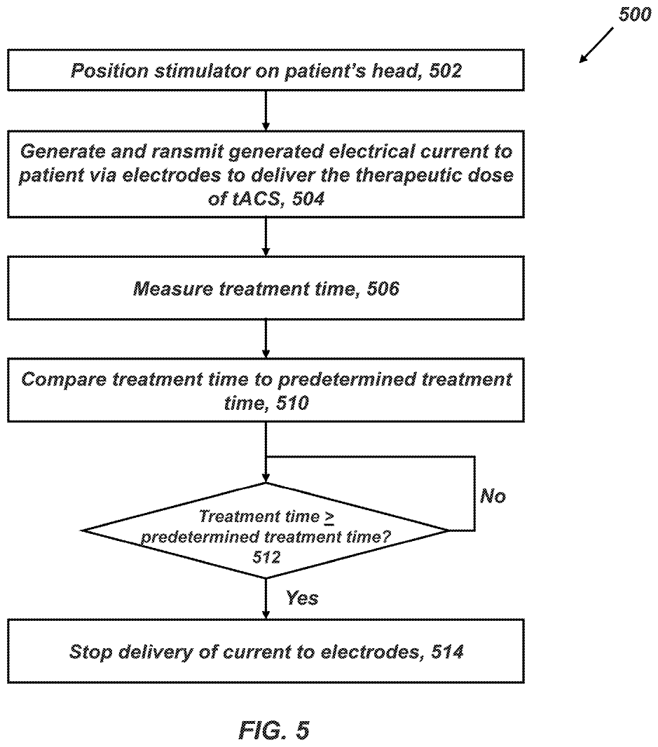

FIG. 5 illustrates a method 500 of treatment using the tACS device of FIG. 1. As shown, embodiments of the method 500 include operations 402-416. However, it can be appreciated that, in alternative embodiments, the method can include greater or fewer operations and the order of one or more of the operations can be varied.

In operation 402, the stimulator is positioned on the patient's head. The patient's scalp and hair should be clean to minimize risk of skin irritation caused by the electrodes. The sponge electrodes should be wet sufficiently to wet the scalp and hair that they are placed in contact with. This ensures proper electrical contact between the electrodes and the patient's head. The wires are electrically connected to the connector at terminals and electrically connected to the sponge electrodes.

The headband is placed over the patient's head so that the strap sits above the patient's eyebrows. The wet electrodes are inserted into the electrode housings with the wires attached. The electrodes are then slid firmly beneath the headband directly up from the sideburns, so that the bottom of the electrode is now lower than the top of the car which is behind and not beneath the electrode. The physiological location of the electrode placement is the squamous temporal bone above the posterior aspect of the zygomatic arch.

In operation 504, once the wet sponge electrodes are in position, the tACS device is turned on (e.g., by rotation of the rotary switch) for generation and transmission of the electrical current to the patient via the electrodes to deliver the therapeutic dose of tACS to the patient. The dial is rotated to go from the OFF to ON position. The standby light should illuminate. Further rotation of the dial causes the fixed current to be generated and delivered to the electrodes. The waveform of the generated current has a predetermined waveform as discussed above, including the carrier frequency and the two modulating frequencies. The amplitude (of the generated current is also fixed at 2.2 mA+/−5%).

In operation 506, the microprocessor measures an amount of time elapsed from start of delivery of the generated electric current (tACS therapeutic dose) to the electrodes. For example, the processor starts a timer (e.g., a 20 minute timer) concurrently with delivery of the generated waveform to the electrodes.

In operation 510, the controller compares an amount of time lapsed from start of the timer to a predetermined therapy time (e.g., 20 minutes). The predetermined therapy time may be retrieved from in a memory in communication with the microprocessor. In an embodiment, the predetermined therapy time can be 20 minutes. However, in alternative embodiments, the predetermined therapy time can adopt other values.

In operation 512, the controller determines if the elapsed time is equal than the predetermined therapy time. If not, the method 500 returns to operation 506. If so, the controller stops output of the waveform to the electrodes at operation 412, ceasing the tACS treatment.

In further embodiments, the tACS device can be configured to determine whether the therapy is interrupted. If so, the timer tracking the therapy can pause and subsequently restart when therapy is resumed (unless the tACS device is turned off). Additionally, under circumstances where the tACS device is turned off, the timer can reset to the predetermined therapy time (e.g., 20 minutes).

The method can further include administering the therapeutic dose of tACS to the patient multiple times daily (e.g., treatment twice daily) for the predetermined treatment time (e.g., about 20 minutes). For example, a first daily treatment can be performed within a predetermined time at the beginning of a patient's daily schedule, such as within two hours after waking from sleep. A second daily treatment can be performed within a predetermined time at the end of a patient's daily schedule, such as within two hours prior to going to sleep.

### Results

### Trial 1-Treatment of Major Depressive Disorder (MDD)

### Overview

A triple-blind, randomized, sham-controlled trial was conducted to demonstrate efficacy of the tACS device embodied herein for treatment of Major Depressive Disorder (MDD) in adults. The patient population of the study were subjects who met the criteria for moderate to severe Major Depressive Disorder (Diagnostic and Statistical Manual of Mental Disorders, Fifth Edition—DSM5), as diagnosed by the study's clinical staff of licensed psychiatrists and psychiatric mental health nurse practitioners. Patients had a baseline Beck Depression Inventory (BDI-II) score between 20 to 63 (moderate to severe).

Participants self-administered treatment at home using either an active tACS device or a sham tACS device for two daily 20-minute treatment sessions, once after waking for the day and a second time before bedtime, for four (4) weeks. The primary outcome was the change in BDI-II score from baseline to the week 2 timepoint in an intent-to-treat analysis, followed by analyses of pre-protocol (“treatment adherent” or “usage compliant”) participants. Secondary analyses examined change at the week 1 and week 4 timepoints, responder rates, subgroup analyses, and safety.

255 participants were randomized to active or sham treatment; 185 female and 70 male participants between the age of 21-65, and residing in 47 US States, were enrolled. In the general population, approximately twice as many females than males are treated for MDD. In the intent-to-treat analysis, significantly greater improvement at week 1 and greater response at week 4 occurred using the tACS device. Importantly, the per protocol analyses determined that active tACS treatment was significantly superior to sham tACS treatment at all time points. Improvements were significantly larger for female participants. Side effects were reported by a small percentage of participants and were mild and temporary.

The study demonstrated that the tACS device discussed herein provides rapid and significant improvement in depression in adults with MDD, particularly for women. Compared to other depression therapies, the tACS device provides advantages of rapid and clinically significant treatment effect, patient self-administration, and the infrequency and minimal nature of adverse events.

Subjects were randomly assigned (approximately 1:1) to active or sham treatment. The subjects, assessors and sponsor were all blinded to study condition (triple blind). The James Blinding Index was used to assess the degree to which participants believed they were using an active device or sham device (subject blinding). The sham device appeared to function like the active device but produced zero electrical output. This study was performed in compliance with good clinical practice (ISO 14155-2020).

Adults meeting DSM5 criteria for MDD, diagnosed by a study clinician, of moderate to severe severity on the Beck Depression Inventory (BDI-II) could participate. Eligibility criteria were: age 21 to 65; US resident; able to read/write English; able to commit to the treatment protocol; no history of suicide attempt or active suicidal ideation with plan or intent in the past 30 days; no previous hospitalization for mental health condition within one year; no use of neuromodulation within one year; no changes in prescription nervous system medication within 30 days; no use of recreational substances, hypnotics, steroids, and/or marijuana products within 30 days; not experiencing problems with alcohol or substance abuse in the past 12 months; not experiencing mental health disorders other than MDD; no known history of heart disease or trigeminal neuralgia; no pacemaker or any form of medical electronics. Sexually active females of childbearing potential were required to commit to practicing at least one method of contraception during trial 1.

All data were entered into the electronic patient-reported outcomes (cPRO) clinical trials software platform managed by the Clinical Research Organization (CRO), Climb Technologies. Potential subjects completed an online pre-screening process. Eligible participants met virtually with investigative staff, who reviewed the trial's purpose, procedures, risks and benefits, compensation, and data confidentiality. Interested participants then completed the informed consent form electronically.

Participants began a 14 day lead-in period (no treatment initiated), after which they retook the self-report assessments and a computer administered M.I.N.I (Mini International Neuropsychiatric Interview (M.I.N.I.): The M.I.N.I. is an assessment tool for the major psychiatric disorders. In the trial, the MINI was used in a participant self-administered format and was confirmed during participants' telemedicine visit with a clinician to determine if any comorbid diagnoses may disqualify the participant from proceeding to the treatment phase of the study.

A BDI-II score between 20 and 63 was required at both initial prescreening and baseline to participate. The goal of this lead-in was to eliminate participants with transient depression that could potentially bias study outcomes. Given the timing of these assessments, participants had to have been experiencing depression for at least one month prior to the start of treatment. During a clinical interview with a clinician, participants were assessed using DSM5 diagnostic criteria for MDD and other psychiatric disorders and M.I.N.I. responses were reviewed for final determination of eligibility.

Eligible participants were randomized to either the active treatment group (active tACS device) or the control group (sham tACS device) by an unblinded investigative staff member, who had no participant contact and did not discuss group allocation with other members of the investigative team. The randomization assignments were made sequentially. There were no restrictions or other inputs dictating randomization order for the initial 250 randomizations required by the protocol. Excess randomizations beyond 250 used a table with equal allocations between active and sham arms in every block of 10.

The active tACS device used by participants in the trial was embodiment of the above-discussed tACS device. The tACS device included an electrical pulse generator, operated by a controller including a processor that produces and transmits an electrical current to two electrodes (or sensor electrodes) in contact with a patient's scalp. The diameter of each electrode was 36 mm. The active tACS device generates a pulsed, alternating, constant current. A third party device testing lab confirmed that the devices produced a current amplitude of between 2.1 mA and 2.2 mA, which was within a ±5% tolerance of 2.2 mA). The device utilized a bidirectional, square (also referred to as rectangular) waveform with a carrier frequency of 15 kHz (+/−5% manufacturing tolerance), a first modulated frequency of 15 Hz (+/−5% manufacturing tolerance), and a second modulated frequency of 500 Hz (+/−5% manufacturing tolerance). The current, delivered with a series of bursts over a burst duration, including an on burst duration of 50 milliseconds and an off burst duration of 16.7 milliseconds, then switched polarity (direction). Each 50 millisecond burst included of a series of sub-bursts. Each sub-burst had a duration of 1 millisecond on duration followed by a 1 millisecond off duration. Each 1 millisecond sub-burst was further included a plurality of pulses. Each pulse had a duration of 33.33 μs (on pulse duration) followed by a 33.33 μs pause (off pulse duration). The active tACS device delivered a predetermined treatment duration of 20 minutes, timed from a memory in communication with the processor, and then turned off automatically, ending the treatment sessions.

Prior to participants commencing the first treatment session, study staff conducted usage training with each participant via video conferencing. The participants assembled the device during the training and practiced using it under staff supervision. Each of the two electrodes were moistened with tap water and positioned on the scalp by placing it under a headband included in the device kit; each electrode was placed on either side of the head, over the squamous temporal bone, located above the posterior aspect of the zygomatic arch. Participants were instructed to self-administer 20-minute treatment twice daily, once after waking for the day, and a second time before bedtime.

After the training session, participants were administered the James Blinding Index, a standardized and validated questionnaire designed to detect the success or failure of participant blinding (whether a participant knows which study arm they are a part of), which asked which type of investigational device they believed they received (active device or control) and selected from five options to indicate the strength of this belief. An analysis of the James Blinding Index results determined that blinding was successful, confirming participants' inability to distinguish an active device from a sham device.

Each day following the device training, subjects reported whether they used the tACS device as instructed and any changes to their health status or medications. After one, two, and four weeks of treatment, participants completed clinical outcome measures and reported any adverse events or changes to concomitant medications.

The Beck Depression Inventory, Second Edition (BDI-II) was the primary instrument used to assess depression severity. The BDI-II is a validated, commonly used, multiple-choice self-report inventory that assesses severity of depression. The BDI-II contains 21 items on a 4-point scale from 0 to 3 for a total score from 0 to 63. The BDI-II is commonly used in clinical research and the mental health field to evaluate psychiatric disorders, and is particularly useful for measuring the effectiveness of treatments that are self-administered by the patient.

### Statistical Analysis

Sample size for trial 1 was based on the primary outcome measure of change in the BDI-II. Sample size calculations were based on a planned sample size of 250 evaluable subjects with 1:1 allocation. Trial 1 provided 80% power, assuming a population mean difference between groups of 3.2 and a common standard deviation of 9, based on a two-sided two-sample t-test with an alpha of 0.05. Previous data suggested the standard deviation for 2-week improvement to be approximately 6.5; however, a conservative estimate of 9 was used for these calculations.

All analyses were performed using SAS® Version 9.4. The primary endpoint analysis was performed at the one-sided 0.025 alpha level, with all other analyses based on a nominal two-sided 0.05 alpha level. Confidence intervals and p-values for secondary endpoints and subgroup analyses were not adjusted for multiple comparisons. The primary analyses used an intent-to-treat (ITT) as a more conservative estimate of treatment effects. Select analyses were then conducted of subjects who reported complete adherence to twice daily treatment, to determine if active treatment was significantly superior to sham treatment when both active group and sham group participants followed usage instructions.

The primary efficacy endpoint was defined as the change in BDI-II score at week 2, as compared to baseline in the active treatment vs. control arm. Analysis was performed using a linear regression model for change, adjusted for each subject's baseline value. Missing data for the primary endpoint only was handled via multiple imputation based on a fully conditional specification with the following covariates: age, sex, baseline BDI-II, week 1 BDI-II, and available follow-up BDI. Imputation was performed separately by treatment group. Several sensitivity analyses were performed. These were supportive in nature and employ nominal confidence levels. The primary endpoint was repeated using the As Treated and Per Protocol (usage adherent) populations. The same statistical methods as those used by the primary endpoint were utilized. Secondary analyses were conducted for the week 1 and 4 timepoints and for the PHQ-9 and QIDS-SR at all timepoints, adjusting for baseline scores. A secondary analysis compared BDI-II responder rate at weeks 1, 2, and 4, with response defined as 50% or greater improvement in score from baseline. Subgroup analysis of the primary endpoint were performed in the ITT analysis set for the subgroups defined by sex, race, and baseline BDI-II (moderate (20-28) vs. severe (29-63) by utilizing an interaction between treatment arm and subgroup. Interaction term with p-value <0.15 were further examined per the pre-specified analysis plan. No adjustment for multiple comparisons was performed. An exploratory analysis examined whether concurrent use of antidepressant medications affected treatment outcome.

### Participant Characteristics

Baseline demographic and clinical characteristics are summarized in Table 1.

TABLE 1
Participant characteristics. Intent to treat sample (N = 255)

| | | Active | | Control | | | | |
|---|---|---|---|---|---|---|---|---|
| | | Treatment | | Treatment | | Total | | |
| Assessment | | N = 126 | | N = 129 | | N = 255 | | p-value |
| Age (year) | N | 126 | | 129 | | 255 | | 0.7182 |
| | Mean | 39.6 | | 40.1 | | 39.8 | | |
| | SD | 10.04 | | 10.90 | | 10.47 | | |
| | Median | 38.5 | | 39 | | 39 | | |
| | Min | 21 | | 21 | | 21 | | |
| | Max | 63 | | 63 | | 63 | | |
| Biological | Female | 96 | (76.19%) | 89 | (68.99%) | 185 | (72.55%) | 0.1978 |
| sex | | | | | | | | |
| (n (n %)) | | | | | | | | |
| | Male | 30 | (23.81%) | 40 | (31.01%) | 70 | (27.45%) | |
| Race/ | White | 92 | (73.02%) | 99 | (76.74%) | 191 | (74.90%) | 0.9280 |
| Ethnicity | | | | | | | | |
| (n (n %)) | | | | | | | | |
| | Asian | 4 | (3.17%) | 5 | (3.88%) | 9 | (3.53%) | |
| | Black or African | 9 | (7.14%) | 7 | (5.43%) | 16 | (6.27%) | |
| | American | | | | | | | |
| | Hispanic or Latino | 9 | (7.14%) | 7 | (5.43%) | 16 | (6.27%) | |
| | Other | 12 | (9.52%) | 11 | (8.53%) | 23 | (9.02%) | |

Based on baseline BDI-II scores. 32.6% and 67.5% of the sample reported moderate and severe depression symptoms, respectively, as shown in Table 2.

TABLE 2
Baseline clinical characteristics

| | | Active | | Control | | | | |
|---|---|---|---|---|---|---|---|---|
| | | Treatment | | Treatment | | Total | | p- |
| Assessment | | N = 126 | | N = 129 | | N = 255 | | value |
| Beck Depression | N | 126 | | 129 | | 255 | | 0.8562 |
| Inventory-II | | | | | | | | |
| (BDI-II) scores | | | | | | | | |
| | Mean | 34.1 | | 33.9 | | 34.0 | | |
| | SD | 8.88 | | 8.99 | | 8.92 | | |
| | Median | 33 | | 33 | | 33 | | |
| | Min | 20 | | 20 | | 20 | | |
| | Max | 57 | | 59 | | 59 | | |
| Beck Depression | Minimal | 0 | (0.00%) | 0 | (0.00%) | 0 | (0.00%) | 0.4244 |
| Inventory-II | | | | | | | | |
| (BDI-II) clinically based | | | | | | | | |
| categories | | | | | | | | |
| | Mild | 0 | (0.00%) | 0 | (0.00%) | 0 | (0.00%) | |
| | Moderate | 44 | (34.92%) | 39 | (30.23%) | 83 | (32.55%) | |
| | Severe | 82 | (65.08%) | 90 | (69.77%) | 172 | (67.45%) | |
| Use of concurrent | | 55 | (43.7%) | 47 | (36.4%) | 102 | (40.0%) | 0.3623 |

anti-depressant medication

The James Blinding Index (0.718, 95% CI [0.668 to 0.768]) showed a lower confidence bound above 0.5 and is considered successful blinding. Overall, 56% and 45% in the active and control groups, respectively, did not believe they knew their assigned treatment. Self-reported adherence was high, with 85.1% of participants reporting twice-daily usage throughout the first 14 days. When broken down by group, 86.5% of subjects in the Active group engaged in complete use through 14 days and 83.7% in the Sham group.

### Outcomes Measures

Tables 3, 4, 5 present the results of the primary and secondary effectiveness endpoints related to the change in BDI-II score in the Per Protocol (Completely Device Use) population.

TABLE 3
Primary efficacy endpoint: Change in BDI-II Score at Week 2

| | | | 95% confidence | |
|---|---|---|---|---|
| Variable | Treatment | Estimate | interval | p-value |
| Change in Beck Depression | Active | 17.15 | 15.040-19.253 | |
| Inventory-II Score at Week 2 | | | | |
| Per Protocol analyses (n = 198) | Control | 13.62 | 11.439-15.802 | |
| | Difference | 3.72 | 1.103-6.340 | 0.005 |
| | Active | 16.65 | 14.691-18.610 | |
| ITT analyses (n = 248) | Control | 14.36 | 12.244-16.476 | |
| | Difference | 2.04 | −0.476-4.549 | 0.056 |

TABLE 4
Change in BDI-II from Baseline to Week 1

| | | | 95% Confidence | |
|---|---|---|---|---|
| Treatment Arm | n | Mean | Interval | p-value |
| Active | 97 | −15.00 | −16.79, −13.21 | |
| Control | 101 | −10.77 | −12.53, −9.02 | |
| Difference | | −4.23 | −6.73, −1.72 | 0.001 |
| (Active-Control) | | | | |

Estimates come from a linear regression model adjusted for baseline BDI-II.
Change = Week 1 − Baseline. Negative numbers indicate improvement.
28 days of 2x use daily.

TABLE 5
Change in BDI-II from Baseline to Week 4

| | | | 95% Confidence | |
|---|---|---|---|---|
| Treatment Arm | n | Mean | Interval | p-value |
| Active | 97 | −20.67 | −22.84, −18.51 | |
| Control | 101 | −16.38 | −18.51, −14.26 | |
| Difference | | −4.29 | −7.32, −1.25 | 0.006 |
| (Active-Control) | | | | |

Estimates come from a linear regression model adjusted for baseline BDI-II.
Change = Week 4 − Baseline. Negative numbers indicate improvement.
28 days of 2x use daily (complete device use).

The analysis of participants with complete usage compliance in the first 14 days showed a significantly greater BDI-II improvement in the active treatment versus control at 2 weeks (difference: 3.72, p=0.005, CI [1.103 to 6.340]), whereas the difference between groups in the Intent to Treat population fell short of statistical significance by six thousands (0.006) of a point (difference of 2.04; one-sided p=0.056, 95% CI [−0.476 to 4.549]). There were significant effects at the secondary 1 week timepoint in the ITT population; the effects in the Per Protocol population were significant at all timepoints. These results validate the tACS device as rapidly effective and safe.

Finally, in responder analyses by week 4, the active treatment group showed a significantly greater responder rate than the control group in the ITT population (65.08% vs. 52.71%, p=0.045), as well as in the Per Protocol population, as shown in Table 6.

TABLE 6
BDI-II responders with at least 50% improvement in BDI-II
by time point Per Protocol (Complete Device Use)

| | | | | |
|---|---|---|---|---|
| Gender | Timepoint | Active Arm | Control Arm | P-Value |

| | | | | | |
|---|---|---|---|---|---|
| All | Week 1 | 40.2% | (39/97) | 26.7% | (27/101) |
| | Week 2 | 53.6% | (52/97)_{—} | 40.6% | (41/101)_{—} |
| | Week 4 | 70.1% | (68/97) | 53.5% | (54/101) |

Concurrent use of antidepressant medication was fairly common, with 43.7% of the active treatment group and 36.4% of the sham group reporting use (Table 2). Within the active treatment group, there were no differences in outcome at week 2 between those on vs. off antidepressant medications (p=0.543).

### Safety Results

The numbers of adverse events were small. 19 subjects in the active group (15.1%) reported 34 events and 10 subjects (7.8%) in the control group reported 13 events. No serious adverse events were reported. Only one AE (skin discomfort) led to device discontinuation.

In conclusion, the trial validated the active tACS device as a rapidly effective and safe treatment for Major Depressive Disorder; additional research is required to establish results in male patients as thoroughly as this study established results in female patients.

### Study 2—Anxiety Treatment

A second trial was conducted to demonstrate effectiveness of the tACS device for treatment of Generalized Anxiety Disorder in First Responders. The tACS devices used in this trial were of the same manufacturing batch as those used in the previously summarized MDD trial. The device specifications and method of use were identical; as in the MDD trial, subjects in the GAD trial were instructed to self-administer the tACS twice a day for 20-minutes, once at the beginning and end of each day.

This was a randomized, waitlist-controlled trial. Upon qualification, participants were randomly assigned into either “immediate” or “deferred” arms. Both arms received an active device and identical treatment instructions. The immediate group began treatment immediately upon enrollment. The waitlisted group (deferred) received treatment after a programed delay of ten (10) days in the shipping logistics process and up to four (4) days in transit (targeted delay of fourteen (14) days). 220 participants were enrolled. A Beck Anxiety Inventory (BAI) score ≥7 will be included as inclusion criteria. Scores and participation were monitored in near-real-time (through the CRO, Climb Technologies) and re-evaluated on a weekly basis. The primary endpoint was the change in the BAI score from baseline to Week 2, comparing the Immediate and Delayed Treatment groups.

### Inclusion Criteria:

1) Read and write English;
2) Employed by First Responder organization;
3) Not have used a “brain stimulation device” in the last year;
4) Commit to 2 (two) 20-minute sessions per day, once in the morning and once in the evening, for up to 8 weeks;
5) Willing to wear a ReadiBand actigraph and share data with the study;
6) Does not have a pacemaker, or electronic stent or have/use any other electronic implant/use regularly for their health;
7) Has not started or is not planning to start any new medical treatment or health program (including new weight loss or fitness program, over the next 8 weeks;
8) Has not contemplated suicide in the past year;
9) Has not been institutionalized for mental health issues;
10) Is not under medical supervision for a serious medical condition (sleep apnea, restless leg syndrome, mental health issues, and/or heart disease);
11) Not currently taking any medications affecting the nervous system (e.g., psychiatric medications);
12) Not allergic to nickel;
13) Not currently taking part or planning to take part over the next 8 weeks in any other study on insomnia, anxiety or other mental health effort (including the NIJ Fatigue Study);
14) Not pregnant or intending to become pregnant in the next 10 weeks;
15) Baseline Beck Anxiety Inventory score >7.

Subjects were randomized into the immediate treatment group, which received active treatment, or the deferred treatment group, which first waited for a period of two weeks before starting active treatment. The Beck Anxiety Inventory was administered to subjects at enrolment, week 2 and week 4. Those who consented for extending treatment were observed until week 8.

Table 7, below, provides the details of subject disposition in trial 2.

TABLE 7
Subject disposition

| Subject disposition | Immediate | | Deferred | |
|---|---|---|---|---|
| Screened | 358 | | | |
| Randomized | 110 | | 110 | |
| Treated* | 105 | (95.5%) | 95 | (86.4%) |
| Discontinued treatment | | | | |
| Loss to follow up | 28 | (26.7%) | 25 | (26.3%) |
| Voluntary withdrawal | 6 | (5.7%) | 7 | (7.4%) |
| Not first responder | 1 | (0.9%) | 0 | |
| Withdrawal due to side effects | 2 | (1.9%) | 1 | (1.1%) |
| Consenting for more than week 4 | 50 | (47.6%) | 51 | (53.7%) |

A total of 358 subjects were screened for the study, out of which 220 fulfilled the inclusion criteria. The eligible subjects were randomized equally in two groups (110 each). In the immediate group, 105 (95.5%) subjects received the treatment, while in the deferred group, 95 (86.4%) subjects had the treatment. At week 4, there were 28 (26.7%) loss to follow subjects in the immediate group, while 25 (26.3%) in the deferred group. There were 6 (5.7%) subjects with voluntary withdrawal in the immediate group, while 7 (7.4%) in the deferred group. Two (1.9%) subjects from the immediate group withdraw due to side effects, while 1 (1.1%) withdraw due to same reason from the deferred group. Among the treated subjects, there were 50 (47.6%) from the immediate group, who showed willingness to continue beyond week 4, while 51 (53.7%) subjects from the deferred group showed the willingness to continue further.

Table 8 shows the descriptive statistics for subject characteristics in two groups.

TABLE 8
Descriptive statistics for subject characteristics

| | | | | | 95% | |
|---|---|---|---|---|---|---|
| | | Group | | Mean | Confidence | P- |
| Characteristics | Statistic | Immediate | Deferred | difference | interval | value |
| Age (years) | N | 110 | 110 | 1.13 | −1.44, 3.69 | 0.388* |
| | Mean | 42.39 | 43.52 | | | |
| | Standard | 9.49 | 9.85 | | | |
| | deviation | | | | | |
| | Median | 41.00 | 41.00 | | | |
| | Minimum | 20.00 | 25.00 | | | |
| | Maximum | 67.00 | 73.00 | | | |
| Gender | Male | 73 (66.4) | 54 (49.1) | | | 0.009^{§} |
| | Female | 37 (33.6) | 56 (50.9) | | | |
| Height (inches) | N | 110 | 110 | −1.37 | −2.47, −0.27 | 0.015* |
| | Mean | 69.45 | 68.07 | | | |
| | Standard | 4.14 | 4.15 | | | |
| | deviation | | | | | |
| | Median | 70.00 | 68.00 | | | |
| | Minimum | 61.00 | 60.00 | | | |
| | Maximum | 80.00 | 80.00 | | | |
| Weight (lbs.) | N | 110 | 110 | −10.42 | −22.75, 1.92 | 0.097* |
| | Mean | 197.90 | 187.48 | | | |
| | Standard | 48.67 | 44.05 | | | |
| | deviation | | | | | |
| | Median | 199.00 | 180.00 | | | |
| | Minimum | 120.00 | 110.00 | | | |
| | Maximum | 350.00 | 310.00 | | | |

*Obtained using t-test for independent samples.
^{§}Obtained using Pearson's chi-square test.
Bold p-values indicate statistical significance.

The mean age of subjects in the immediate group was 42.39 years (SD: 9.49 years), while that in the deferred group was 43.52 years (SD: 9.85 years), and the difference of means was statistically not significant (p=0.388). Regarding gender, there were 66.4% males in the immediate group, as against 49.1% in the deferred, while the female proportion was lower in the immediate group (33.6%) as compared to the deferred group (50.9%). The gender distribution was significantly different in two groups as indicated by a p-value of 0.009. The mean overall height of subjects in the immediate group was 69.45 inch (SD: 4.14 inch), while that of the deferred group was 68.07 inch (SD: 4.15 inch), and the difference in the means was statistically significant with a p-value of 0.015. The mean weight of subjects in the immediate group was 197.90 lbs. (SD: 48.67 lbs.), while in the deferred group was 187.48 lbs. (44.05 lbs.), and the difference of means was statistically not significant (p=0.097).

Table 9 gives the descriptive statistics for Beck Anxiety Inventory (BAI) score in two groups at different time points.

TABLE 9
Descriptive statistics for Beck Anxiety Inventory
score in two groups according to time

| | | Group | | |
|---|---|---|---|---|
| Visit | Statistic | Immediate | Deferred | P-value* |
| Baseline | N | 105 | 95 | 0.905 |
| | Mean | 20.14 | 19.80 | |
| | Standard Deviation | 10.45 | 9.40 | |
| | Median | 19.00 | 18.00 | |
| | Minimum | 1.00 | 5.00 | |
| | Maximum | 48.00 | 43.00 | |
| Week 2 | N | 81 | 87 | <0.001 |
| | Mean | 12.79 | 19.24 | |
| | Standard Deviation | 7.86 | 9.29 | |
| | Median | 11.00 | 18.00 | |
| | Minimum | 0.00 | 5.00 | |
| | Maximum | 44.00 | 45.00 | |
| Week 4 | N | 73 | 77 | 0.024 |
| | Mean | 10.64 | 13.08 | |
| | Standard Deviation | 6.93 | 7.10 | |
| | Median | 10.00 | 11.00 | |
| | Minimum | 0.00 | 0.00 | |
| | Maximum | 39.00 | 35.00 | |

*Obtained using Mann-Whitney U test.
Bold p-values indicate statistical significance.

At baseline, the parameter violated the normality assumption, as per Shapiro-Wilk's test, and hence non-parametric evaluation was performed to compare the scores between two groups. At baseline, the mean BAI score for the immediate group was 20.14 (SD: 10.45) and median of 19, while for the deferred group was 19.80 (SD: 9.40) and median of 18. The difference of median scores was statistically not significant (p=0.905). At week 2, the mean BAI in the immediate group was 12.79 (SD: 7.86) and median of 11, while in the deferred group, the mean was 19.24 (SD: 9.29) and median of 18. The difference in the median BAI score of two groups at week 2 was statistically significant with a p<0.001. Further at week 4, the mean for the immediate group was 10.64 (SD: 6.93) with a median of 10, while for the deferred group was 13.08 (SD: 7.10) with a median of 11, and the difference of medians was statistically significant with a p-value of 0.024.

Table 10 provides the descriptive statistics for the change in BAI scores from baseline to week 2.

TABLE 10
Comparison of change in BAI scores (D) from
baseline to week 2 between two groups

| | | Group | | |
|---|---|---|---|---|
| | Statistic | Immediate | Deferred | P-value |
| Baseline to | N | 81 | 87 | <0.001 |
| Week 2 | Mean | 6.33 | 0.38 | |
| | Standard Deviation | 7.83 | 7.67 | |
| | Median | 5.00 | 0.00 | |
| | Minimum | −11.00 | −29.00 | |
| | Maximum | 29.00 | 29.00 | |

*Obtained using Mann-Whitney U test.
Bold p-values indicate statistical significance.

The comparison of change in scores revealed that for week 2, the mean change in score in the immediate group was 6.33 (SD: 7.83) and a median of 5, while that in the deferred group was 0.38 (SD: 7.67) and a median of 0. The difference of medians between groups was statistically significant with a p<0.001.

Table 11 is the cross-table showing frequencies of subjects in each BAI category at baseline and week 2 for the immediate group.

TABLE 11
Change in anxiety level from baseline to week 2 in
immediate group based on BAI score categorization

| | | BAI category: Week 2 | | | |
|---|---|---|---|---|---|
| | | <= 21 | 22-35 | >=36 | |
| IMMEDIATE | | (Low) | (Moderate) | (Severe) | Total |
| BAI category: | <=21 (Low) | 53 | 1 | 0 | 54 (66.7%) |
| Baseline | | | | | |
| | 22-35 (Moderate) | 13 | 7 | 0 | 20 (24.7%) |
| | >=36 (Severe) | 4 | 2 | 1 | 7 (8.6%) |
| Total | | 70 (86.4%) | 10 (12.3%) | 1 (1.2%) | 81 |

P < 0.001 using Marginal homogeneity test

There were 54 (66.7%) subjects in low category at baseline, out of which 53 continued to be in low category at week 2, while 1 moved to moderate category. There were 20 (24.7%) subjects in moderate category at baseline, out of which 13 moved to low category, 7 continued to be moderate. Further, out of 7 (8.6%) subjects with severe category at baseline, 4 moved to low category, 2 to moderate and 1 continued with severe category. At week 2, there were 70 (86.4%) subjects in the low category, 10 (12.3%) in moderate and 1 (1.2%) in severe category.

The marginal distribution changed significantly from baseline to week 2, as indicated by p<0.001 with significantly higher proportion of subjects in low category at week 2 as compared to baseline.

Table 12 is the cross-table showing frequencies of subjects in each BAI category at baseline and week 2 for the deferred group.

TABLE 12
Change in anxiety level from baseline to week 2 in
deferred group based on BAI score categorization

| | | BAI category: Week 2 | | | |
|---|---|---|---|---|---|
| | | <=21 | 22-35 | >=36 | |
| DEFERRED | | (Low) | (Moderate) | (Severe) | Total |
| BAI category: | <=21 (Low) | 46 | 6 | 1 | 53 (60.9%) |
| Baseline | | | | | |
| | 22-35 (Moderate) | 7 | 18 | 3 | 28 (32.2%) |
| | >=36 (Severe) | 2 | 3 | 1 | 6 (6.9%) |
| Total | | 55 (63.2%) | 27 (31.0%) | 5 (5.7%) | 87 |

P = 0.590 using Marginal homogeneity test

There were 53 (60.9%) subjects in low category at baseline, out of which 46 continued to be in low category at week 2, while 6 moved to moderate category and 1 to severe category. There were 28 (32.2%) subjects in moderate category at baseline, out of which 7 moved to low category, 18 continued to be moderate and 3 moved to severe category. Further, out of 6 (6.9%) subjects with severe category at baseline, 2 moved to low category, 3 to moderate and 1 continued with severe category. At week 2, there were 55 (63.2%) subjects in low category, 27 (31.0%) in moderate and 5 (5.7%) in severe category. The marginal distribution did not change significantly from baseline to week 2 in the deferred group (p=0.590).

The willingness to continue beyond four weeks was indicated by 53 subjects from the immediate group and 50 from the deferred group. Accordingly, a subset analysis was performed on these subjects and the parameters were compared at week 8 between the two groups, as shown in Table 13.

TABLE 13
Descriptive statistics for different
scores at week 8 in two study groups

| | | Group | | |
|---|---|---|---|---|
| Parameter | Statistic | Immediate | Deferred | P-value |
| Week 8-BAI score | N | 53 | 50 | 0.010* |
| | Mean | 8 | 11.72 | |
| | Standard | 6.47 | 7.96 | |
| | Deviation | | | |
| | Median | 6 | 10 | |
| | Minimum | 0 | 0 | |
| | Maximum | 35 | 28 | |

*Obtained using Mann-Whitney U test.
Bold p-values indicate statistical significance.

Table 13 reveals that the BAI score demonstrated a difference of medians/means between two groups (p>0.05) at week 8. The BAI score at week 8 in the immediate groups was significantly smaller than that of the deferred group (p=0.010).

Accordingly, this study achieved its primary endpoint and validates the tACS device as rapidly effective for the treatment of Generalized Anxiety Disorder (GAD).

Comparative Study—Disqualifying 1 mA of tACS as a Therapeutic Dose

In 2015, the results of an MDD study conducted at Harvard Medical School determined that using a previous version of the tACS device that produced an average amplitude of 1 mA (half of the amplitude of the tACS embodied herein), and administering 1 mA tACS to patients only once per day, five days per week, was ineffective. Following is the study abstract.

“We examined efficacy and safety of one specific cranial electrical stimulator (CES) device at a fixed setting in subjects with treatment-resistant major depressive disorder (MDD). Thirty subjects (57% female, mean age 48.1±12.3 years) with MDD and inadequate response to standard antidepressants were randomized to 3 weeks of treatment with CES (15/500/15,000 Hz, symmetrical rectangular biphasic current of 1-4 mAmp, 40 V) or sham CES (device off) for 20 min, 5 days per week. The primary outcome measure was improvement in the 17-item Hamilton Depression Rating Scale (HAM-D-17). Adverse effects (AEs) were assessed using the Patient Related Inventory of Side Effects (PRISE). Completion rates were 88% for CES, 100% for sham. Both treatment groups demonstrated improvement of about 3-5 points in HAM-D-17 scores (p<0.05 for both), and no significant differences were observed between groups. Remission rates were 12% for CES, and 15% for sham, a nonsignificant difference. CES was deemed safe, with good tolerability; poor concentration and malaise were the only distressing AEs that differed significantly between CES and sham (p=0.019 and p=0.043, respectively). Limitations include a small sample and lack of an active comparator therapy. Although both treatment groups improved significantly, this CES at the setting chosen did not separate from sham in this sample. Thus we cannot rule out that the benefit from this setting used in this particular form of CES was due to placebo effects. Since this form of CES has other settings, future studies should test these settings and compare it against other CES devices.”

In conclusion, this comparative study disqualifies 1 mA of tACS, administered once per day for five days per week, for the treatment of MDD. The MDD and GAD studies summarized above validated use of embodiments of the tACS device embodied herein, which target 2.2 mA of average amplitude (+/−5% manufacturing tolerance) and uses twice-daily treatment, seven days per week.

Certain exemplary embodiments have been described to provide an overall understanding of the principles of the structure, function, manufacture, and use of the systems, devices, and methods disclosed herein. One or more examples of these embodiments have been illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

The subject matter described herein can be implemented in analog electronic circuitry, digital electronic circuitry, and/or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value or a manufacturing tolerance. For example, the value specified may vary by ±10%, ±9%, ±8%, ±7%, ±6%, ±5%, ±4%, ±3%, ±2%, ±1%, ±0.9%, ±0.8%, ±0.7%, ±0.6%, ±0.5%, ±0.4%, ±0.3%, ±0.2%, ±0.1%. For example, the manufacturing tolerance can be ±5%. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

1. A method of transcranial alternating current (tACS) stimulation, comprising:
generating and transmitting, by a controller including a processor, an electrical current for receipt by two electrodes in contact with opposing lateral sides of a patient's scalp to deliver a therapeutic dose of tACS for a predetermined treatment time;
wherein the generated electrical current has a constant, an average amplitude of about 2.2 mA and a bidirectional square waveform utilizing a carrier waveform having a frequency of about 15 kHz, a first modulating waveform having a frequency of about 15 Hz, and a second modulating waveform having a frequency of about 500 Hz;
wherein the generated current includes a plurality of sub-bursts within a burst duration, the burst duration including a burst on duration of about 50 ms including the plurality of sub-bursts followed by a burst off duration of about 16.7 ms without the plurality of sub-bursts;
wherein the current switches polarity after each burst duration;
wherein each sub-burst includes a plurality of pulses within a sub-burst duration, the sub-burst duration including a sub-burst on duration of 1 ms including the plurality of pulses followed by a sub-burst off duration of about 1 ms without the plurality of pulses;
wherein each pulse of the plurality of pulses extends for about 33.33 μs followed by a pause of about 33.33 μs.

2. The method of claim 1,
wherein each of the two electrodes is positioned on the squamous temporal bone, above the posterior aspect of the zygomatic arch and maintained in place during delivery of the therapeutic dose of tACS for the treatment of Major Depressive Disorder (MDD) or Generalized Anxiety Disorder (GAD);
wherein the diameter of each of the electrodes is about 36 mm; and
further comprising retrieving, by the processor, the predetermined treatment time from a memory in communication with the processor, wherein the predetermined treatment time is about 20 minutes;
measuring a treatment time during which the therapeutic dose of tACS is delivered to the patient;
comparing the treatment time to the predetermined treatment time; and
ceasing transmission of the generated electrical current when the elapsed time is equal to the predetermined treatment time.

3. The method of claim 2, further comprising administering the therapeutic dose of tACS to the patient twice daily for the predetermined treatment time, wherein one administration occurs within two hours or less after waking from sleep and another administration occurs within two hours or less prior to sleeping.
